(19) United States
(12) Patent Application Publication
(10) Pub. No.: US 2023/0202861 A1
(43) Pub. Date: Jun. 29, 2023

(54) SYNTHESIS OF NANOPARTICLES

(71) Applicant: UCL BUSINESS LTD, London (GB)

(72) Inventors: Maximilian BESENHARD, London (GB); Luca PANARIELLO, London (GB); Liudmyla STOROZHUK, London (GB); Alec LAGROW, London (GB); Asterios GAVRIILIDIS, London (GB); Thanh Thi Kim NGUYEN, London (GB)

(73) Assignee: UCL BUSINESS LTD, London (GB)

(21) Appl. No.: 17/928,457

(22) PCT Filed: May 28, 2021
(86) PCT No.: PCT/GB2021/051323

§ 371 Date: Nov. 29, 2022

(30) Foreign Application Priority Data
May 29, 2020 (GB) 2008101.4

(51) Int. Cl. C01G 49/00; B01J 19/06; B01J 19/00; A61K 49/18

(52) U.S. Cl. CPC C01G 49/0027; B01J 19/06; B01J 19/0093; A61K 49/1887; C01P 2004/64; C01P 2006/44; C01P 2004/04; C01P 2002/72; B82Y 40/00

(57) ABSTRACT

A method for synthesising metal oxide nanoparticles. The method comprises mixing, to provide a reaction mixture, a precursor solution comprising metal ions with an initiator solution to initiate a nanoparticle precipitation process, and then quenching the precipitation process by adding a quenching agent to the reaction mixture so as to yield a dispersion comprising metal oxide nanoparticles. The resulting metal oxide nanoparticles may have an average diameter of less than 7 nm, for example 5 nm or less.

### FIELD OF THE INVENTION

The invention relates to the synthesis of metal oxide nanoparticles, particularly magnetic metal oxide nanoparticles such as ferrite nanoparticles.

### BACKGROUND

The use of magnetic metal oxide nanoparticles, particularly iron oxide nanoparticles (IONPs), as T₁ MRI contrast agents is a current area of interest due to the health risks posed by existing contrast agents such as gadolinium complexes. IONPs are a potential substitute as they are biocompatible and high T₁ effects have been observed for very small nanoparticles. The rationale for the good T₁ performance of small IONPs lies in their high surface-to-volume ratio, which allows for an increased exposure of iron (Fe) ions to surrounding water. However, the reproducible and scalable production of biocompatible IONPs of a small enough size remains challenging.

Particularly for biomedical applications, such as the synthesis of MRI contrast agents, water-based synthesis of magnetic metal oxide nanoparticles is preferred over thermal decomposition synthesis methods despite the advantages for size and shape control associated with thermal decomposition. This is because thermal decomposition requires high boiling point solvents and ligand exchange steps after synthesis. This is one of the reasons why the most commonly used synthesis of IONPs, otherwise known as ferrite nanoparticles, is the co-precipitation of iron salts in aqueous solutions, i.e., the simultaneous precipitation of ferrous (Fe′) and ferric ions (Fe′) by mixing with a base, commonly sodium hydroxide or ammonium hydroxide. Other reasons for the popularity of the co-precipitation synthesis are the cheap starting materials, simple experimental procedures that are performed at moderate temperatures (less than 100° C.), and the lack of toxic educts or by-products.

However, co-precipitation syntheses, particularly batch synthesis methods, are known to yield particles of a relatively low magnetisation and high polydispersity of size due to variations in IONP core sizes and the formation of aggregates. Especially the latter is a problem for colloidal stability (larger particles will sediment in a solution as the Brownian movement of the surrounding water molecules is not sufficient to keep them suspended). The polydispersity is believed to originate from simultaneous nucleation and growth of particles and the occurrence of intermediate phases before or during the formation of the desired magnetic phases, i.e., magnetite (Fe₃O₄) or maghemite (γ-Fe₂O₃). These are the most magnetic iron oxide phases and both of these phases are therefore particularly suitable for use as MRI contrast agents.

There is still no common knowledge of the exact nanoparticle formation mechanism when using precipitation synthesis methods. In general though, nanoparticle precipitation processes are thought to comprise an initial nucleation phase in which nanoparticles are initially formed, followed by a growth phase during which the nanoparticles increase in size. The initially formed (or nucleated) nanoparticles (sometimes referred to as nanometre-sized primary particles) may not necessarily comprise the metal oxide phases that are ultimately desired (e.g. magnetite/maghemite) and these phases may be formed later in the process from initially formed intermediate phases. For example, in the case of synthesis of IONPs, the initially nucleated nanoparticles may comprise other phases such as intermediate iron oxides, iron hydroxides, and/or iron(oxy)hydroxide phases (FeₓOₓOHₓHₓ), and the desired crystalline metal oxide phases (e.g. magnetite/maghemite) may be formed later in the process after initial nucleation following supply of metal ions from primary particles comprising amorphous metal oxide phases. Fluctuations in mixing time alter the intermediate, and hence the final, IONP properties, as well as the nucleation kinetics. Such rapid phase changes complicate the reproducible and reliable synthesis of monodisperse particles having a well-defined size.

There is therefore a need for a method for synthesising metal oxide nanoparticles, particularly magnetic metal oxide nanoparticles such as ferrite nanoparticles, that is capable of reliably providing colloidally stable, monodisperse dispersions of nanoparticles of a controllable, small size in a cost-effective and scalable manner that requires a minimum number of processing steps. In particular, there is a need for a method for synthesising small magnetic nanoparticles that are suitable for use as contrast agents in magnetic resonance imaging (MRI).

### SUMMARY OF THE INVENTION

According to a first aspect, the invention provides a method for synthesising metal oxide nanoparticles. The method comprises mixing, to provide a reaction mixture, a precursor solution comprising metal ions with an initiator solution to initiate a nanoparticle precipitation process; and quenching the precipitation process by adding a quenching agent to the reaction mixture so as to yield a dispersion comprising metal oxide nanoparticles.

The dispersion may be a colloidal dispersion.

The quenching agent may inhibit the precipitation process. More specifically, the quenching agent may inhibit growth of the metal oxide nanoparticles, for example following initial nanoparticle nucleation. For example, the quenching agent may dissolve precipitates or nanoparticles (e.g. primary particles) comprising intermediate phases so as to inhibit the further growth of previously formed metal oxide nanoparticles comprising magnetic metal oxide phases such as magnetite/maghemite or other spinel-structure phases. The quenching agent may therefore be added to the reaction mixture to quench or inhibit nanoparticle growth once nanoparticles comprising or consisting of the desired magnetic or spinel-structure metal oxide phases have been formed.

The metal oxide nanoparticles in the colloidal dispersion may have an average diameter of less than about 8 nm, for example as measured by TEM. Preferably, the metal oxide nanoparticles in the colloidal dispersion have an average diameter of less than about 7 nm, more preferably less than about 6 nm, for example about 5 nm or less.

The quenching agent may be added to the reaction mixture within about 100 seconds of (i.e. after) the initiation of the precipitation process.

The quenching agent may be added to the reaction mixture within about 10 seconds of the initiation of the precipitation process.

The quenching agent may be added to the reaction mixture once nanoparticles comprising, consisting of, or consisting essentially of magnetic metal oxide phases have been formed. For example, the quenching agent may be added to the reaction mixture once nanoparticles comprising, consisting of, or consisting essentially of spinel structure metal oxide phases have been formed. This is because spinel structure phases tend to have particularly good magnetic properties. The quenching agent may be added to the reaction mixture once nanoparticles comprising, consisting of, or consisting essentially of magnetite and/or maghemite have been formed.

The quenching agent may be added to the reaction mixture more than about 3 seconds after the initiation of the precipitation process.

For example, the quenching agent may be added to the reaction mixture within about 100 seconds or 10 seconds of the initiation of the precipitation process and more than about 3 seconds after the initiation of the precipitation process.

The quenching agent may be added to the reaction mixture about 5 seconds after the initiation of the precipitation process.

The metal oxide nanoparticles may be magnetic. The metal oxide nanoparticles may comprise, consist of, or consist essentially of magnetic metal oxide phases, such as spinel structure metal oxide phases. The metal oxide nanoparticles may comprise, consist of, or consist essentially of magnetite and/or maghemite. The metal oxide nanoparticles may be substantially free of non-magnetic metal oxide phases. The metal oxide nanoparticles may be substantially free of non-spinel structure metal oxide phases. For example, for IONPs the nanoparticles may be substantially free of iron oxide phases that are not magnetite and/or maghemite.

The metal oxide nanoparticles may comprise or may be ferrite nanoparticles.

The metal ions may comprise iron ions, such as ferrous and/or ferric ions, particularly when the desired metal oxide nanoparticles are iron oxide nanoparticles (IONPs). The metal ions may alternatively be other ions, depending on the nanoparticles that are to be synthesised.

The initiator solution may be an alkaline solution. The initiator solution may comprise at least one base. For example, the initiator solution may comprise sodium hydroxide and/or ammonium hydroxide. The alkaline solution may alternatively or additionally comprise one or more other bases, such as potassium hydroxide (KOH), tetramethylammonium hydroxide (TMAOH), tetraethylammonium hydroxide (TEAOH), sodium carbonate (Na₂CO₃), and/or Urea. The base is preferably a strong base, such as sodium hydroxide, ammonium hydroxide, potassium hydroxide (KOH), tetramethylammonium hydroxide (TMAOH), or tetraethylammonium hydroxide (TEAOH).

The quenching agent may comprise at least one acid. The at least one acid may comprise citric acid and/or hydrochloric acid.

The at least one acid may comprise or may be a chelating agent.

Adding the quenching agent to the reaction mixture may lower the pH of reaction mixture. For example, adding the quenching agent to the reaction mixture may lower the pH of reaction mixture sufficiently to quench the precipitation process, specifically to quench or inhibit growth of the metal oxide nanoparticles. For example, the quenching agent may lower the pH of the reaction mixture to about pH 8 or less.

The quenching agent may be added to the reaction mixture so as to substantially neutralise the pH of the reaction mixture. The quenching agent may be added to the reaction mixture so as to acidify the reaction mixture, i.e. to reduce the pH of the reaction mixture below 7.

The colloidal dispersion comprising the metal nanoparticles may be a colloidally stable dispersion. For example, the adding of the quenching agent to the reaction mixture may yield a colloidally stable dispersion of or comprising the metal oxide nanoparticles. The reaction mixture may comprise one or more stabilisers to stabilise the nanoparticle dispersion. One or more stabilisers may therefore be added to the reaction mixture. For example, the quenching solution may comprise one or more stabilisers. Alternatively or additionally the precursor solution and/or the initiator solution may comprise one or more stabilisers. The one or more stabilisers may comprise one or more carboxylic acids (e.g. citric acid), dextran, polyacrylic acid, and/or polyethylene glycol (PEG).

The colloidal dispersion comprising the metal oxide nanoparticles may be substantially monodisperse in size.

The method may be performed in a flow reactor.

The mixing of the precursor solution with the initiator solution may occur at a first junction or mixer (e.g. static mixer) of the flow reactor.

The addition of the quenching agent to the reaction mixture may occur at a second junction or mixer (e.g. static mixer) of the flow reactor downstream from the first junction.

The residence time of the reaction mixture in the flow reactor between the first and second junctions may be equal to the quenching delay time, where the quenching delay time is the time between initiation of the precipitation process and quenching of the precipitation process by addition of the quenching agent.

According to another aspect, the invention provides use of metal oxide nanoparticles produced by the method of the invention as an MRI contrast agent.

According to another aspect, the invention provides an MRI contrast agent comprising metal oxide nanoparticles produced by the method of the invention.

According to another aspect, the invention provides a catalyst comprising metal oxide nanoparticles produced by the method of the invention.

According to another aspect, the invention provides a flow reactor configured to perform the method of the invention.

The flow reactor may comprise a first junction or mixer (e.g. static mixer) configured to mix, thereby forming a reaction mixture, a precursor solution comprising metal ions with an initiator solution to initiate precipitation of metal oxide nanoparticles.

The flow reactor may comprise a second junction or mixer (e.g. static mixer) arranged downstream of the first junction configured to mix the reaction mixture with a quenching solution comprising a quenching agent so as to quench the precipitation process and thereby yield a dispersion comprising the metal oxide nanoparticles.

The flow reactor may be configured such that the residence time of the reaction mixture in the flow reactor between the first and second junctions is equal to the quenching delay time.

The flow reactor may comprise a first reservoir configured to supply the precursor solution to the first junction. The first reservoir may contain the precursor solution.

The flow reactor may comprise a second reservoir configured to supply the initiator solution to the first junction. The second reservoir may contain the initiator solution.

The flow reactor may comprise a third reservoir configured to supply the quenching solution to the second junction. The third reservoir may contain the quenching solution.

Also disclosed is a method for making an MRI contrast agent, the method comprising synthesising metal oxide nanoparticles according the method described herein, and then preparing an MRI contrast agent incorporating the metal oxide nanoparticles.

Also disclosed is a method for making a catalyst, the method comprising synthesising metal oxide nanoparticles according the method described herein, and then preparing a catalyst incorporating the metal oxide nanoparticles.

### BRIEF DESCRIPTION OF THE FIGURES

The invention will now be described, by way of example only, with reference to the accompanying drawings, in which:

FIG. 1 shows a schematic of a flow reactor that may be used in accordance with the invention;

FIG. 2A shows a TEM image of Sample A of Example 1;

FIG. 2B shows a TEM image of Sample B of Example 1;

FIG. 3 shows the results of SQUID magnetization measurements performed on Sample E of Example 3; and

FIG. 4 shows an XRD spectrum of Sample E of example 3.

FIG. 5a shows a histogram of the particle size distribution for the nanoparticles synthesised in Example 4a.

FIG. 5b shows a histogram of the particle size distribution for the nanoparticles synthesised in Example 4b.

FIG. 5c shows a histogram of the particle size distribution for the nanoparticles synthesised in Example 4c.

### DETAILED DESCRIPTION

The following description is intended to introduce various aspects and features of the invention in a non-limiting manner. For clarity and brevity, features and aspects of the invention may be described in the context of particular embodiments. However, it should be understood that features of the invention that are described only in the context of one or more embodiments may be employed in the invention in the absence of other features of those embodiments, particularly where there is no inextricable functional interaction between those features. Even where some functional interaction between the features of an embodiment is discernible, it is to be understood that those features are not inextricably linked if the embodiment would still fulfil the requirements of the invention without one or more of those features being present. Thus, where features are, for brevity, described in the context of a single embodiment, those features may also be provided separately or in any suitable sub-combination. It should also be noted that features that are described in the context of separate aspects and embodiments of the invention may be used together and/or be interchangeable wherever possible. It is also to be understood that all disclosed optional features, values and ranges may be combined with any other optional features, values and ranges in any suitable combination and it should therefore be understood that all such combinations are therefore disclosed. For example, individual features may be extracted from a plurality of lists of features and combined, and all such combinations are to be understood to be disclosed herein.

Features described in connection with the invention in different contexts (e.g. method, use, device etc.) may each have corresponding features definable and/or combinable with respect to each other, and these embodiments are specifically envisaged.

The method of the invention exploits the finding that the initial nucleation step of the precipitation process occurs on a very short timescale, as does the formation of the desired magnetic metal oxide phases such as magnetite and maghemite, and that it is possible to quench the subsequent growth of the metal oxide nanoparticles by the rapid addition of a quenching agent, such as an acid or acidic solution, so as to arrest or inhibit further growth of the nanoparticles. Controlled quenching of the precipitation process in this way, specifically quenching growth of the metal oxide nanoparticles once the desired magnetic metal oxide phases are formed, has been found to yield magnetic metal oxide nanoparticles having a small size that are suitable for use as MRI contrast agents. The quenching agent may also inhibit agglomeration and aggregation of the metal oxide nanoparticles, thereby avoiding the sedimentation often observed following precipitation synthesis of nanoparticles.

The initially formed (nucleated) nanoparticles (which are sometimes referred to as nanometre-sized primary particles) may not necessarily comprise the metal oxide phases that are ultimately desired (e.g. magnetite/maghemite) as these phases may be formed later in the process from initially formed intermediate phases. For example, in the case of synthesis of IONPs, the initially nucleated nanoparticles may comprise other phases such as intermediate iron oxides, iron hydroxides, and/or iron(oxy)hydroxide phases (FeₓOₓOHₓHₓ), and the desired crystalline metal oxide phases (e.g. magnetite/maghemite) may be formed later in the process after initial nucleation following supply of metal ions from primary particles comprising amorphous metal oxide phases. The intermediate phases are more soluble than the desired magnetic metal oxide phases and the quenching agent may dissolve precipitates or nanoparticles (e.g. primary particles) comprising intermediate phases so as to inhibit the further growth of the metal oxide nanoparticles comprising magnetic metal oxide phases such as magnetite/maghemite. The quenching agent may therefore be added to the reaction mixture to quench or inhibit nanoparticle growth once nanoparticles comprising or consisting essentially of magnetic or spinel-structure metal oxide phases have been formed.

The quenching agent therefore quenches growth of the metal oxide nanoparticles during the precipitation process following initial nucleation of the nanoparticles. The invention therefore provides a means for providing small metal oxide nanoparticles of a controlled and relatively uniform size. The resulting metal oxide nanoparticle dispersion may also be colloidally stable. In this sense, colloidally stable means that the nanoparticles remain substantially dispersed or suspended at equilibrium under ambient conditions (e.g. at room temperature (25° C.) and atmospheric pressure), with substantially no sedimentation of the nanoparticles occurring even when left for an extended period of time, for example over a period of one or more months.

The smaller size of the nanoparticles (in particular their higher surface area to volume ratio) and their colloidal stability means that the resulting nanoparticles are suitable for use as MRI contrast agents, and in particular T₁ contrast agents. The nanoparticles of the invention may therefore be used as contrast agents. The nanoparticles may also be used in other applications where a high surface area to volume ratio is advantageous, such as catalysis. The nanoparticles may therefore be used as, or in the manufacture of, a catalyst.

It is surprising that rapid addition of a quenching agent at a precise time shortly after initiation of the precipitation process provides the advantages of providing small (e.g. an average diameter of ≤5 nm) metal oxide nanoparticles comprising or consisting of the desired magnetic metal oxide phases. The quenching step of the invention is performed before completion of the precipitation process so as to arrest or halt (i.e. quench) the precipitation process, namely the growth of the nanoparticles, and prevent further nanoparticle growth and/or agglomeration. This is distinct from stabilisation or de-agglomerisation of nanoparticles once the nanoparticle precipitation process has substantially concluded, which does not yield the same advantageous results. The method of the invention is particularly effective when using a strong base (e.g. NaOH) to initiate the precipitation process. This causes the initial nucleation step and the formation of magnetic metal oxide phases to proceed rapidly, resulting in formation of magnetic nanoparticles within a short timescale of a few second or so. The quenching agent is then added to inhibit the growth of the nanoparticles following formation of these magnetic metal oxide phases. The quenching agent may therefore be added following initial nucleation and once the magnetic phases are formed, but before the end of (i.e. during) the subsequent growth phase.

It is surprising that the desired magnetic metal oxide phases are formed so early in the precipitation process. For example, when synthesising IONPs magnetite and maghemite are surprisingly still formed even when performing early quenching, and that the desirable magnetic properties of the nanoparticles are therefore maintained, and even enhanced, following quenching. Quenching therefore advantageously inhibits further particle growth and agglomeration while at the same time allowing the formation of these desirable magnetic phases, which were generally believed to be formed only later during the precipitation process.

It has also been found that the method of the invention, particularly when performed using a flow reactor, is highly scalable and is capable of yielding large amounts of nanoparticles in a relatively short timescale. For example, the synthesis of several grams or even kilograms of nanoparticles per hour is possible. The cost is also relatively low.

The method of the invention involves forming metal oxide nanoparticles by precipitation from an aqueous precursor solution comprising metal ions. The metal oxide nanoparticles may be magnetic metal oxide nanoparticles such as ferrite (i.e. iron oxide) nanoparticles. Ferrite nanoparticles, otherwise known as iron oxide nanoparticles (IONPs), may comprise other metals in addition to iron, such as zinc, manganese, or magnesium. For example, the IONPs may be zinc- or manganese-doped IONPs or they may be doped with other metals. The IONPs may therefore be doped IONPs. In the following, where a specific metal or type of metal oxide nanoparticle is referred to, such as iron or IONPs, it should be understood that other suitable metals or metal oxides could be used instead, particularly magnetic metal oxides.

Typically, the metal ions in the aqueous solution are provided by one or more dissolved metal salts. The aqueous solution comprising metal ions may therefore be an aqueous solution of (i.e. comprising) one or more metal salts. For example, in the case of the synthesis of IONPs the salts may be one or more iron salts comprising ferrous and/or ferric ions.

The precipitation process is initiated by mixing an aqueous precursor solution comprising metal ions with an initiator solution. The initiator solution generally comprises a base and is generally an alkaline solution. The initiator solution is therefore a source of hydroxide ions. The mixing of the initiator solution with the precursor solution results in an increase of the pH of the precursor solution which initiates the precipitation of metal oxide nanoparticles. In other words, mixing the initiator solution with the precursor solution yields a reaction mixture having a higher pH than the precursor solution, and it is the increased pH of the reaction mixture relative to the precursor solution that initiates the precipitation process. IONPs, for example, may be synthesised via the co-precipitation of iron salts in aqueous solution. More specifically, ferrous and ferric ions may be co-precipitated in the presence of a base according to the following reaction scheme:

2Fe³⁺+Fe²⁺+8OH⁻→Fe₃O₄+4H₂O.

This may be achieved by mixing an aqueous precursor solution comprising ferrous and ferric ions with an aqueous initiator solution comprising a base such as sodium hydroxide or ammonium hydroxide, e.g. an alkaline solution. More generally, depending on the precise details of the precipitation process and the type of metal oxide nanoparticles being formed, the initiator solution may alternatively or additionally comprise one or more other bases, such as potassium hydroxide (KOH), tetramethylammonium hydroxide (TMAOH), tetraethylammonium hydroxide (TEAOH), sodium carbonate (Na₂CO₃), and/or Urea. The identity of the base may therefore be selected as required. However, as explained above, the base is preferably a strong base (i.e. one that is fully dissociated in aqueous solution), such as sodium hydroxide, ammonium hydroxide, potassium hydroxide, tetramethylammonium hydroxide, or tetraethylammonium hydroxide as this results in rapid nucleation and controlled quenching of the growth phase by the quenching agent. In general, the pH of the reaction mixture that results from mixing of the precursor and initiator solutions is preferably greater than or equal to pH 6, as this results in rapid nucleation and rapid formation of the desired magnetic metal oxide phases. For example, for the synthesis of IONPs the pH may be raised to about pH 12 or greater after mixing the precursor and initiator solutions. Alternatively, the pH may be increased less than this, for example to greater than or equal to pH 4 or 5.

Once the precursor solution and the initiator solution have been mixed together to form a reaction mixture, thereby initiating the precipitation process, a quenching agent is subsequently added to the reaction mixture in order to quench the precipitation process, specifically the growth (including colloidal growth mechanisms) of nanoparticle nuclei. The quenching agent is added while the precipitation process is ongoing, i.e. before the precipitation process is substantially completed, so as to quench or arrest the precipitation process. The quenching agent is preferably added in the form of an aqueous quenching solution comprising the quenching agent. The quenching agent inhibits the precipitation process, thereby preventing or inhibiting the formation of larger nanoparticles. The quenching agent preferably comprises one or more acids, and the quenching solution is therefore preferably an acidic solution. For example, the quenching agent may comprise a carboxylic acid, such as citric acid. Alternatively or additionally, the one or more acids may comprise an inorganic acid such as hydrochloric acid.

The quenching solution may also comprise one or more stabilisers to stabilise the nanoparticle dispersion. Alternatively, one or more stabilisers may be added to the reaction mixture in another way. For example, the precursor and/or initiator solution may comprise one or more stabilisers or stabilising agents. Stabilisers stabilise the metal oxide nanoparticles. Many nanoparticle stabilisers are known, and examples include chelating agents such as carboxylic acids (e.g. citric acid). Stabilisers may alternatively contain other functional groups that have a preferred interaction or an affinity with the metal (e.g. iron) of the metal oxide, either as metal ions in solution or on the surface of the nanoparticles, or both. Examples of such stabilisers including dextran, polyacrylic acid, and/or polyethylene glycol (PEG). By adding one or more stabilisers to the precursor and/or initiator solution it is possible to reduce the resulting metal oxide nanoparticle sizes further, for example so that the nanoparticles have an average diameter of ≤4 nm, or even ≤3 nm. This is because the stabilisers act to slow the precipitation process, including the growth phase, even before the quenching solution is added to quench the precipitation process. Even small amounts of stabiliser are sufficient to reduce the size of the resulting nanoparticles. For example, the stabiliser may be added to the reaction mixture so that the molar ratio of the stabiliser to the metal ions in the reaction mixture is at least 0.05:1, but lower amounts of stabiliser may be added to the reaction mixture.

The quenching agent may comprise or may be a chelating agent. For example, citric acid is a chelating agent, as are other carboxylic acids comprising a plurality of carboxyl groups. Chelating agents are particularly effective quenching agents because the chelating agent helps to prevent further nanoparticle nucleation due to the chelation of the metal ions with the chelating agent. Chelating agents also chelate with the metal oxide nanoparticles, thereby stabilizing the metal oxide nanoparticles and inhibiting their growth to a degree. The quenching agent may therefore comprise an acid that is also a chelating agent.

Adding the quenching agent typically reduces the pH of the reaction mixture sufficiently to quench the precipitation process. For example, the quenching agent may lower the pH of the reaction mixture to about pH 8 or less. The quenching agent may, for example, substantially neutralise the reaction mixture. For example, the addition of the quenching agent may bring the pH of the reaction mixture within a range of from pH 6 to pH 8, more preferably within a range of from pH 6.5 to pH 7.5. The neutralisation of the reaction mixture inhibits the precipitation process, which relies on alkaline conditions and the availability of hydroxide ions to proceed. Neutralisation of the reaction mixture is generally sufficient when using a quenching agent such as citric acid. Neutralisation is not essential though, and stable nanoparticles can be formed at lower pH values. In particular, stable nanoparticles have been observed following quenching by hydrochloric acid to yield a reaction mixture having a pH in the range of 2 to 4. The quenching agent may therefore instead be added to the reaction mixture so as acidify the reaction mixture, i.e. to lower the pH of the reaction mixture below pH 7.

The quenching solution may have a lower temperature than the reaction mixture so as to induce rapid cooling of the reaction mixture once it is added. This acts to further enhance the quenching effect of the quenching agent. For example, the quenching solution may be at least 5 or 10° C. cooler than the reaction mixture. Alternatively or additionally, the reaction mixture may be cooled, preferably rapidly, either before, or preferably simultaneously with or following addition of the quenching agent, for example by immersing the channels of the reactor in a cold medium such as a water bath. The cooling is preferably performed substantially contemporaneously with the addition of the quenching agent. For example, the cooling may occur within 1 second either before or after addition of the quenching agent. The temperature of the reaction mixture may be reduced by at least 5 or 10° C., for example.

As mentioned above, the quenching agent is added before the precipitation process is substantially completed, and therefore interrupts the precipitation process. More specifically, the quenching agent quenches the nanoparticle growth process that follows initial nucleation, and the quenching agent is therefore added before the growth phase is substantially complete. For example, the quenching agent may be added/fed to the reaction mixture within about 100 seconds after the mixing of the precursor solution with the initiator solution, i.e. after initiation of the precipitation process. More preferably, the quenching agent may be added/fed to the reaction mixture within about 10 seconds, after the mixing of the precursor solution with the initiator solution. It has been found that adding the quenching agent at very short times following initiation of the precipitation process may result in the formation of non-magnetic phases, which is undesirable for certain applications. To avoid this, the quenching agent should therefore be added a sufficient time after the initial mixing of the precursor solution and the initiator solution such that the metal oxide nanoparticles comprise substantially magnetic phases, in other words so that the nanoparticles are substantially free from non-magnetic metal oxide phases. It is therefore preferably to add the quenching agent to the reaction mixture more than 2 seconds, preferably more than 3 seconds, after initiation of the precipitation process. These values are particularly illustrative of the typical range of times that have been found to yield colloidally stable, monodisperse IONP dispersions having acceptably small nanoparticles. However, other timescales are possible, depending on the end result desired and the reagents/conditions used. In general though, the quenching agent is added to quench the growth of the nanoparticles once magnetic metal oxide phases have been formed, in other words once nanoparticles comprising or consisting essentially of magnetic metal oxide phases have been formed.

The resulting dispersion preferably comprises metal oxide nanoparticles that are magnetic. The dispersion may therefore comprise metal oxide nanoparticles that comprise, consist of, or consist essentially of magnetic metal oxide phases, such as spinel structure metal oxide phases. The dispersion may therefore comprise metal oxide nanoparticles that comprise, consist of, or consist essentially of magnetite and/or maghemite. The metal oxide nanoparticles may be substantially free of non-magnetic metal oxide phases. The metal oxide nanoparticles may be substantially fee of non-spinel structure metal oxide phases. For example, for IONPs the nanoparticles may be substantially free of iron oxide phases that are not magnetite and/or maghemite.

Compared to conventional precipitation synthesis methods, the method of the invention yields nanoparticles having a smaller size. Conventional co-precipitation methods typically yield nanoparticles having average diameters of more than 8 nm. The method of the invention, on the other hand, typically yields metal oxide nanoparticles having smaller average diameters of less than about 8 nm, preferably less than about 7 nm, more preferably less than about 6 nm, for example about 5 nm or less. Nanoparticles having these sizes, particularly those having an average diameter of about 5 nm or less, have been shown to be particularly good MRI contrast agents. These quoted diameters may be measured by transmission electron microscopy (TEM), for example from an analysis of TEM image data, and are calculated as a number average. In other words, the diameter may be the mean (number average) particle diameter as measured from one or more TEM images of the nanoparticles. For example, the areas of a plurality of nanoparticles (e.g. at least 100 nanoparticles) may be measured (e.g. using image processing software such as ImageJ). This may be done by fitting a polygon around the edges of the nanoparticles. The average diameter of the nanoparticles may be determined by averaging (i.e. taking the mean value) of the circle of equal projection area of the measured nanoparticles. The diameters may therefore refer to the (mean) circle of equal projection area of the nanoparticles as measured using TEM. The diameters of the resulting nanoparticles can be tailored for a given set of reaction conditions by appropriately adjusting the quenching time (otherwise known as the quenching delay time), i.e. the time between the initial mixing of the precursor solution and the initiator solution (i.e. initiation of the precipitation process) and the addition of the quenching agent to the reaction mixture. The method may therefore comprise quenching the precipitation process at a selected quenching time by adding a quenching agent to the reaction mixture so as to yield a dispersion comprising the metal oxide nanoparticles, the quenching time being selected such that the metal oxide nanoparticles in the dispersion have a certain diameter, for example any of the diameters mentioned above. Similarly, in applications where magnetic nanoparticles are desired (such as MRI contrast agents) it is preferable for the quenching time to be selected to be sufficiently long so that the resulting nanoparticles comprise substantially magnetic metal oxide phases.

The precipitation process is preferably carried out at an elevated temperature, i.e. above room temperature. In other words, the reaction mixture, and preferably also the reagents, namely the aqueous solution of metal ions, the alkaline solution, and the quenching solution, are at an elevated temperature. For example, the precipitation process may be performed at a temperature in the range of 25 to 90° C., for example at about 60° C.

It has also been found that in order to reliably control the particle size, the initial mixing of the precursor solution with the initiator solution should be performed rapidly (i.e. the mixing process itself should be swift, and ideally as close to instantaneous as possible), as should the subsequent addition of the quenching agent. This provides for consistent reaction conditions (e.g. spatial variations in pH are minimised), ensuring consistent reaction kinetics and dynamics. This also ensures a controlled and uniform reaction time before rapid quenching occurs, thereby checking (e.g. stopping or halting) the growth of the nanoparticles when they are of a uniform size, resulting in a substantially monodisperse nanoparticle size distribution. Such rapid mixing and controlled reaction conditions may be provided by performing the method in a flow reactor. Preferably, the flow reactor is a millifluidic or microfluidic flow reactor, which has the advantage of providing near instantaneous mixing of the various reagent and quenching solutions and therefore provides for an enhanced level of control over the precipitation and quenching processes.

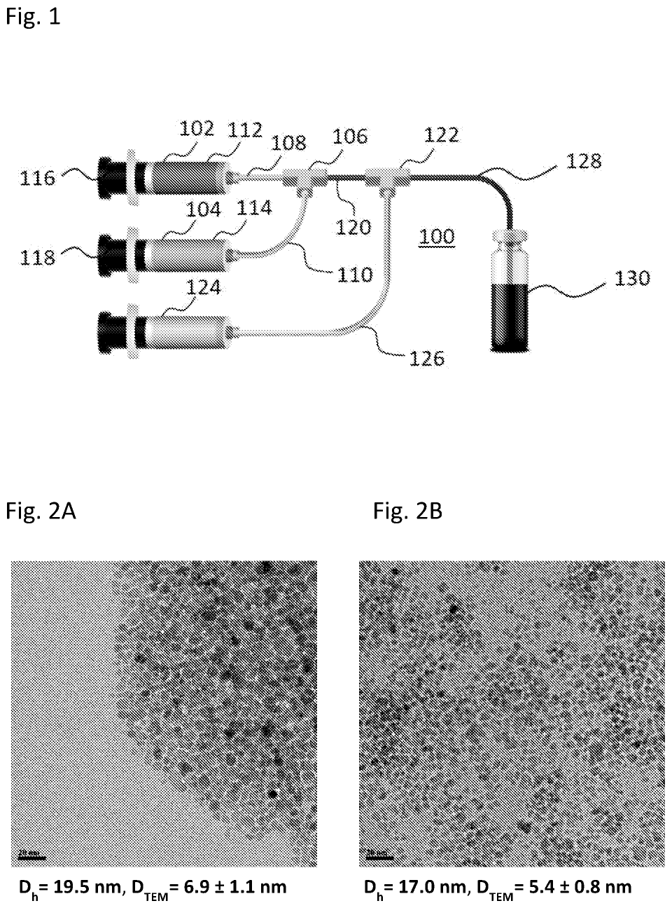

Referring to FIG. 1, the flow reactor 100 may comprise a first reservoir 102 containing the precursor solution and a second reservoir 104 containing the initiator solution. The flow reactor may also comprise a first mixer junction 106 (otherwise known as a mixer), such as a T-junction or other static mixer, arranged to mix together the precursor solution and the initiator solution provided from the first 102 and second 104 reservoirs, respectively. The first mixer junction 106 may therefore be arranged in fluid communication with the first 102 and second 104 reservoirs via first 108 and second 110 reagent supply lines (e.g. tubes) through which the precursor and initiator solutions may flow, respectively, from the reservoirs 102, 104 to the first junction 106. The flow reactor is arranged to push or pump the precursor solution and the initiator solution from the first 102 and second 104 reservoirs, respectively, to the first junction 106. For example, the first 102 and second 104 reservoirs may each comprise a canister 112, 114 and a piston 116, 118 arranged to reduce the volume of the reservoir 102, 104 as it is advanced within the canister 112, 114, thereby forcing the solution to be pushed out of the reservoir 102, 104 and along the respective reagent supply tube 108, 110 to the first mixer junction 106. The precursor solution and the initiator solution are mixed together at the first junction 106, thereby initiating the precipitation process, specifically initiating nucleation of the nanoparticles. Other methods of pumping the solutions from the reservoirs are also possible, and syringe pumps comprising a canister and a piston are mentioned only as an example.

The reaction mixture that results from the mixing together of the precursor solution and the initiator solution then flows along a reaction line 120 or tube that provides for flow communication between the first mixing junction 106 and a second mixer junction 122, which may also be a T-junction or other static mixer. As the reaction mixture advances along the reaction tube 120 the precipitation process proceeds, resulting in growth of the nucleated nanoparticles and formation of the desired metal oxide phases, such as crystalline magnetic metal oxide phases. The quenching agent is then added to the reaction mixture at the second mixing junction 122 in the form of a quenching solution comprising the quenching agent, which is supplied to the second mixing junction 122 from a third reservoir 124 via a quenching agent supply line 126. The third reservoir 124 may be substantially similar to the first 102 and second 104 reservoirs. The reaction mixture and the quenching solution are mixed together at the second mixing junction 122, where after the resulting quenched reaction mixture may flow along a product line 128 or tube, and may subsequently be collected in a product reservoir 130, such as a vial.

The mixing together of the precursor and initiator solutions and the addition of the quenching solution at the mixing junctions 106, 122 provides for very rapid, near instantaneous mixing. This means that the initiation of the precipitation process and the quenching occur at well-defined times relative to each other for any given portion of the reaction mixture as it flows through the flow reactor. In a flow reactor set-up the time between the initiation of the precipitation process (i.e. the mixing of the precursor and initiator solutions) and quenching (i.e. the mixing of the quenching solution with the reaction mixture) is the flow time of the reaction mixture between the first 106 and second 122 mixing junctions. This is also referred to as the residence time of the reaction mixture in the flow reactor between the first 106 and second 122 mixing junctions. The addition time of the quenching solution relative to the initial mixing of the reagent solutions may also be referred to as the quenching or quenching delay time. Thus, although the synthesis of nanoparticles occurs continuously in a flow reactor, with the reagent solutions being continuously mixed at the first mixing junction and the quenching solution being continuously mixed at the second junction, there is still a time delay for any given portion of the reaction mixture between the initiation of the precipitation process and the addition of the quenching agent to the reaction mixture, i.e. the quenching time or quenching delay time. This quenching time may be precisely controlled by adjusting the flow rate of the reaction mixture between the first 106 and second 122 mixing junctions and/or by adjusting the flow distance between the first 106 and second 122 mixing junctions. The residence time of the reaction mixture in the flow reactor between the first 106 and second 122 mixing junctions may therefore be equal to any of the times discussed above defining the time difference between the initiation of the precipitation process and the addition of the quenching agent. For example, the residence time may be less than the time taken for the precipitation process to reach completion, and may be, for example, 100 seconds or less, more preferably 10 seconds or less, for example 7 seconds or less. The residence time may also be more than 3 seconds. For example, the residence time may be about 5 seconds.

The resulting nanoparticles, particularly IONPs, may be used as magnetic resonance imaging (MRI) contrast agents, particularly T₁ contrast agents. The method of the invention may therefore comprise using at least a portion of the resulting magnetic metal oxide nanoparticles as an MRI contrast agent.

### Example 1

IONPs were synthesised in a flow reactor according to the following procedure. An aqueous solution comprising FeCl₃ at a concentration of 0.07 M (mol/L) and FeCl₂ at a concentration of 0.03 M was mixed at a first junction (P-246 T-mixer, IDEX Health Science) of the flow reactor with an aqueous alkaline solution comprising NaOH at a concentration of 0.57 M in equal volumes (a 1:1 volume ratio) to form a reaction mixture. Both reagent solutions were at a temperature of about 60° C. The aqueous solutions were each supplied to the first junction along a respective supply line having an internal diameter of 1 mm at a flow rate of 5 ml/min. The reaction mixture was then flowed between the first junction and a second junction (P-716 T-mixer, IDEX Health Science) of the flow reactor along a tube having an inner diameter of 1 mm at a flow rate of 10 ml/min while being held at about 60° C. An aqueous quenching solution comprising citric acid at a concentration of 0.32 M (also at a temperature of about 60° C.) was then mixed with the reaction mixture at the second junction of the flow reactor at a volume ratio of 2.1:10 (i.e. 2.1 ml of quenching solution to each 10 ml of reaction mixture). The reaction mixture was then flowed through a further 15 m of tubing with an inner diameter of 1.5 mm to allow for a further residence time in the flow reactor of several minutes at about 60° C. The resulting IONP dispersion was then collected in a vial at room temperature (approx. 22° C.) for analysis.

FIG. 2A shows a TEM image of Sample A prepared by the above method in which the residence time of the reaction mixture in the flow reactor between the first and second mixing junctions was set to 100 seconds, by which time the precipitation process is substantially complete and the quenching agent is added too late to quench the precipitation process. FIG. 2B shows a TEM image of Sample B prepared by the above method in which the residence time of the reaction mixture in the flow reactor between the first and second mixing junctions was set to 5 seconds. Sample B therefore demonstrates the effect of quenching the precipitation process, which is still ongoing at a time of 5 seconds following the mixing of the reagent solutions.

Further samples synthesised by the same method but with the residence time between the first and second junctions set to 1 to 3 seconds were found to yield phases other than the desired magnetic IONP phases and separation was difficult.

It can clearly be seen from FIGS. 2A and 2B that the IONPs of Sample B are less agglomerated and smaller in size than those of Sample A. The number average (mean) nanoparticle diameters of the samples were measured from the TEM images as follows. The areas of at least 100 nanoparticles were measured using image processing software (ImageJ) by fitting a polygon around the edges of the nanoparticles. Reported diameters refer to the (mean) circle of equal projection area of the measured nanoparticles. The TEM-measured diameter of the IONPs in Sample A (D_{TEM}) was 6.9±1.1 nm, whereas the TEM-measured diameter of the IONPs in Sample B was 5.4±0.8 nm. The errors reported are the standard deviation. The lower standard deviation of Sample B demonstrates that the IONPs of Sample B have a lower dispersity of diameter than those of Sample A.

The smaller size of the IONPs in Sample B compared to Sample A was also borne out by hydrodynamic diameter measurements (Dₕ) of the samples. The Dₕ of the IONPs was measured via dynamic light scattering. The measured Dₕ of Sample A was 19.5 nm, whereas the Dₕ of Sample B was 17.0 nm, i.e. 2.5 nm less. The hydrodynamic diameter is the diameter of a hypothetical hard sphere that diffuses in the same manner as the particle being measured. The hydrodynamic diameter is therefore larger than the actual diameter of the nanoparticles due to solvation effects, but it is nevertheless clear that the IONPs of Sample B have a smaller diameter than those of Sample A.

### Example 2

IONP Samples C and D were synthesised as per the method set out above for Example 1. The residence time (quenching delay time) was set to 120 s for Sample C and 5 s for sample D. The IONP D_{TEM} diameters were measured as per Example 1, and a relaxometric analysis was performed on the samples to determine the relaxivity of the IONPs. The relaxivity of an MRI contrast agent reflects how the relaxation rates of a solution change as a function of concentration [C] of the contrast agent. Since a contrast agent may affect the two relaxation rates (1/T₁ and 1/T₂) individually, there are two corresponding relaxivities, denoted r1 and r2, where:

1/ΔT₁=r₁·[C] and 1/ΔT₂=r₂·[C]

The relaxation rates of a contrast agent in solution are obtained by graphing changes in relaxation rates (1/ΔT₁) and (1/ΔT₂) at different concentrations. The gradients represent r₁ and r₂. The relaxivity measurements of the IONPs were obtained at a temperature of 37° C., a field strength of 1.41 T (60 MHz), and in water. The results are shown in Table 1.

TABLE 1

| Sample | D_{TEM} (nm) | r₁ (mM⁻¹s⁻¹) | r₂ (mM⁻¹s⁻¹) | r₂/r₁ |
|---|---|---|---|---|
| C | 7-8 | 10.09 | 43.35 | 4.30 |
| D | 5 | 9.61 | 32.60 | 3.34 |

It can again be seen from Table 1 that the IONP diameters that result from quenching (Sample D) are substantially smaller than those following late addition of the quenching agent (Sample C). It can also be seen from Table 1 that the r₂/r₁ ratio of Sample D is lower than that of Sample C, making it particularly well suited to use as a T₁ contrast agent in MRI.

### Example 3

IONP Sample E was prepared using a similar procedure to Example 1, except that an aqueous solution of 1M hydrochloric acid at a flow rate of 1.9 mL/min was used as the quenching agent instead of the citric acid solution. Stable IONPs were again observed following addition of the quenching agent at a quenching delay time of 5 s with a measured D_{TEM} of approx. 7 nm.

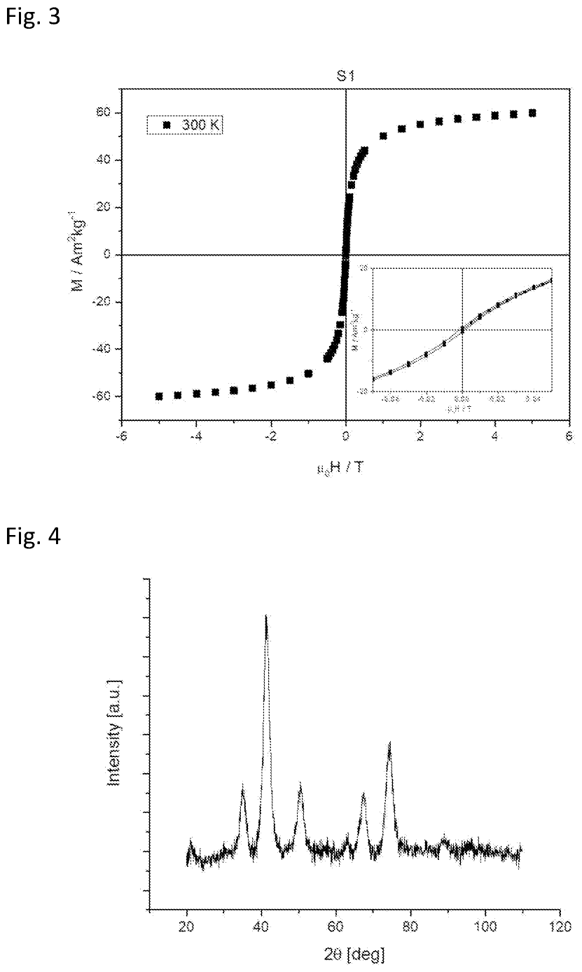

SQUID (superconducting quantum interference device) magnetization measurements were performed on Sample E, the results of which are shown in FIG. 3. The SQUID measurements gave a magnetization in line with superparamagnetic magnetite/maghemite IONPs.

An XRD (X-ray powder diffraction) spectrum was also obtained, as shown in FIG. 4. The XRD spectrum matches that of pure magnetite/maghemite.

It is therefore clear that the desired magnetic phases are still formed even following early quenching of the precipitation process.

### Example 4

### Example 4a

IONPs were synthesised in a flow reactor in accordance with the invention. Precursor and initiator solutions were mixed at a first junction of the flow reactor and a quenching solution was added to the resulting reaction mixture at a second junction of the flow reactor downstream of the first junction. The flow rates and compositions of the precursor solution, initiator solution, and quenching solution were as set out in Table 2a. The residence time between the first and second mixing junctions (the quenching delay time) was set to 5 s. All of the solutions were mixed at 60° C. and held at 60° C. throughout. The residence time of the reaction mixture in the flow reactor between the first junction and collection was 3 min.

TABLE 2a

| | FeCl₂•4H₂O | FeCl₃•6H₂O | NaOH | Citric acid | Flow rate |
|---|---|---|---|---|---|
| | [M] | [M] | [M] | [M] | [ml/min] |
| Precursor | 0.0355 | 0.0710 | 0 | 0 | 5 |
| solution | | | | | |
| Initiator | 0 | 0 | 0.57 | 0 | 5 |
| solution | | | | | |
| Quenching | 0 | 0 | 0 | 0.316 | 2.1 |
| solution | | | | | |

### Example 4b

IONPs were synthesised in a flow reactor in accordance with the invention, with the flow rates and compositions of the precursor solution, initiator solution, and quenching solution as set out in Table 2b. The reaction set-up and conditions were otherwise identical to Example 4a.

TABLE 2b

| | FeCl₂•4H₂O | FeCl₃•6H₂O | NaOH | Citric acid | Flow rate |
|---|---|---|---|---|---|
| | [M] | [M] | [M] | [M] | [ml/min] |
| Precursor | 0.0355 | 0.0710 | 0 | 0.0059 | 5 |
| solution | | | | | |
| Initiator | 0 | 0 | 0.57 | 0 | 5 |
| solution | | | | | |
| Quenching | 0 | 0 | 0 | 0.316 | 2.0 |
| solution | | | | | |

### Example 4c

IONPs were synthesised in a flow reactor in accordance with the invention, with the flow rates and compositions of the precursor solution, initiator solution, and quenching solutions as set out in Table 2c. The reaction set-up and conditions were otherwise identical to Example 4a.

TABLE 2c

| | FeCl₂•4H₂O | FeCl₃•6H₂O | NaOH | Citric acid | Flow rate |
|---|---|---|---|---|---|
| | [M] | [M] | [M] | [M] | [ml/min] |
| Precursor | 0.0355 | 0.0710 | 0 | 0.0500 | 5 |
| solution | | | | | |
| Initiator | 0 | 0 | 0.57 | 0 | 5 |
| solution | | | | | |
| Quenching | 0 | 0 | 0 | 0.316 | 1.5 |
| solution | | | | | |

Results

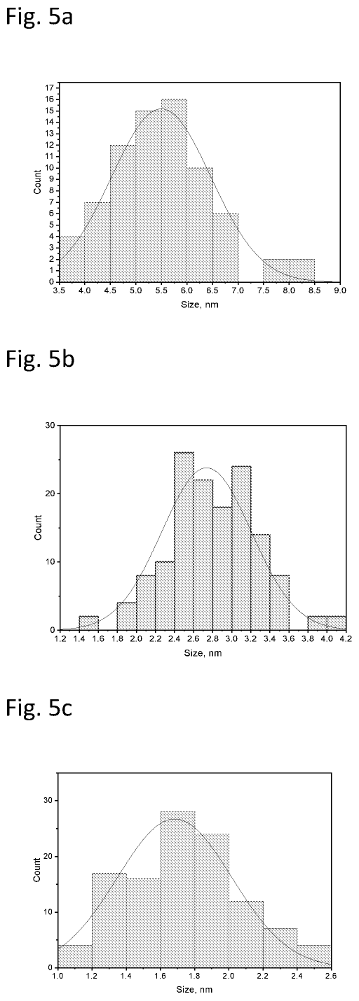

FIG. 5a is a histogram of the particle size distribution for the nanoparticles synthesised in Example 4a, FIG. 5b is a histogram of the particle size distribution for the nanoparticles synthesised in Example 4b, and FIG. 5c is a histogram of the particle size distribution for the nanoparticles synthesised in Example 4c (where the “size” on the x-axis is the TEM-measured diameter, D_{TEM}).

It can clearly be seen from FIGS. 5a-c that the addition of a stabiliser, in this case citric acid, to the reaction mixture through addition of the stabiliser to the precursor solution results in a noticeable reduction in the average nanoparticle diameter. The average (mean) TEM diameter, D_{TEM}, of the synthesised nanoparticles was measured as 5.4±0.5 nm for Example 4a (no stabiliser), 2.7±0.5 nm for Example 4b (0.0059 M citric acid), and 1.7±0.3 nm for Example 4c (0.05 M citric acid), demonstrating a notable reduction in the size of the nanoparticles upon addition of the stabiliser, even in relatively small amounts.

Project Funding

The project leading to this patent application has received funding from the European Union's Horizon 2020 research and innovation programme under the Marie Skłodowska-Curie grant agreement No 721290.

The project leading to this patent application has also received funding from the Engineering and Physical Sciences Research Council (EPSRC grant number EP/M015157/1).

1. A method for synthesising metal oxide nanoparticles, the method comprising:
mixing, to provide a reaction mixture, a precursor solution comprising metal ions with an initiator solution to initiate a nanoparticle precipitation process; and
quenching the precipitation process by adding a quenching agent to the reaction mixture so as to yield a dispersion comprising metal oxide nanoparticles.

2. The method of claim 1, wherein the quenching agent quenches the precipitation process by inhibiting growth of the metal oxide nanoparticles.

3. The method of claim 1, wherein the metal oxide nanoparticles in the dispersion have an average diameter of less than 7 nm.

4. (canceled)

5. The method of claim 1, wherein the quenching agent is added to the reaction mixture within about 100 seconds of the initiation of the precipitation process.

6. (canceled)

7. The method of claim 1, wherein the quenching agent is added to the reaction mixture once nanoparticles comprising magnetic metal oxide phases have formed.

8. The method of claim 1, wherein the quenching agent is added to the reaction mixture more than about 3 seconds after the initiation of the precipitation process.

9. The method of claim 1, wherein the metal oxide nanoparticles are magnetic.

10. The method of claim 1, wherein the metal oxide nanoparticles are ferrite nanoparticles.

11. The method of claim 10, wherein the metal ions comprise iron ions.

12. The method of claim 1, wherein the initiator solution comprises a base.

13. The method of claim 1, wherein the quenching agent comprises at least one acid.

14. The method of claim 13, wherein the at least one acid is a chelating agent.

15. The method of claim 13, wherein the at least one acid comprises citric acid and/or hydrochloric acid.

16. The method of claim 1, wherein adding the quenching agent reduces the pH of reaction mixture so as to inhibit growth of the metal oxide nanoparticles.

17. The method of claim 1, wherein the precursor solution and/or the initiator solution comprises one or more stabilisers.

18. The method of claim 1, wherein the dispersion comprising the metal nanoparticles is a colloidally stable dispersion.

19. The method of claim 1, wherein the method is performed in a flow reactor.

20. The method of claim 19, wherein the mixing of the precursor solution with the initiator solution occurs at a first junction of the flow reactor and the addition of the quenching agent to the reaction mixture occurs at a second junction of the flow reactor downstream from the first junction.

21. (canceled)

22. (canceled)

23. A product comprising metal oxide nanoparticles produced by the method of claim 1, wherein the product is an MRI contrast agent or a catalyst.

24. (canceled)

25. A flow reactor configured to perform the method of claim 1.
